(19) United States
(12) Patent Application Publication
(10) Pub. No.: US 2023/0106731 A1
(43) Pub. Date: Apr. 6, 2023

(54) INHIBITORS OF MUTANT FAMILY TYROSINE-KINASES

(71) Applicant: Spectrum Pharmaceuticals, Inc., Irvine, CA (US)

(72) Inventors: Prasad V. Chaturvedula, Cheshire, CT (US); Prasad Kolli, Mission Viejo, CA (US)

(73) Assignee: Spectrum Pharmaceuticals, Inc., Irvine, CA (US)

(21) Appl. No.: 18/057,239

(22) Filed: Nov. 21, 2022

Related U.S. Application Data
(63) Continuation of application No. 16/757,072, filed on Apr. 17, 2020.
(60) Provisional application No. 62/574,110, filed on Oct. 18, 2017.

(51) Int. Cl. A61K 31/517; C07D 239/94; A61K 31/69; A61K 31/506

(52) U.S. Cl. CPC A61K 31/517; C07D 239/94; A61K 31/69; A61K 31/506

(57) ABSTRACT

An epidermal growth factor receptor (EGFR) family tyrosine kinase inhibitor comprising a functional group that can bind to the serine S797 residue in EGFR having a C797S mutation or the serine S805 residue in HER2 having a C805S mutation.

### FIELD OF THE DISCLOSURE

The disclosure relates generally to inhibitors of mutant epidermal growth factor receptor (EGFR) family tyrosine kinases, pharmaceutically acceptable salts or solvates thereof, and pharmaceutical compositions comprising the EGFR family tyrosine kinase inhibitors, salts or solvates thereof as active ingredients. More particularly, the disclosure relates to EGFR family tyrosine kinase inhibitors selective for a C797S mutant in EGFR and/or a C805S mutant in HER2 which effectively inhibits the growth of cancer cells induced by the overexpression or activation of EGFR family tyrosine kinases.

### BACKGROUND

There are many signal transduction systems in cells which are functionally linked to each other to control the proliferation, growth, metastasis and apoptosis of cells. The breakdown of the intracellular controlling system by genetic and environmental factors causes abnormal amplification or destruction of the signal transduction system leading to tumor cell generation.

Protein tyrosine kinases play important roles in such cellular regulation and their abnormal expression or mutation has been observed in cancer cells. The protein tyrosine kinase is an enzyme which catalyzes the transportation of phosphate groups from ATP to tyrosines located on protein substrates. Many growth factor receptor proteins function as tyrosine kinases to transport cellular signals. The interaction between growth factors and their receptors normally can control cellular growth, but abnormal signal transduction caused by the mutation or overexpression of any of the receptors often induces tumor cells and cancers.

Protein tyrosine kinases have been classified into many families in accordance with their growth factor types, and tyrosine kinases that are structurally related to epidermal growth factor receptor (EGFR), in particular, have been intensely studied. An EGFR tyrosine kinase is composed of a receptor and tyrosine kinase, and delivers extracellular signals to cell nucleus through the cellular membrane. The EGF receptor tyrosine kinase family includes EGFR (Erb-B1), HER2 (Erb-B2), HER3 (Erb-B3), and Erb-B4, each of which can form a homodimer- or heterodimer-signal delivery complex. Also, the overexpression of more than one such heterodimers is often observed in malignant cells. In addition, it is known that both EGFR and HER2 significantly contribute to the formation of heterodimer-signal delivery complexes.

Activating mutations in the kinase domain of EGFR, for example, are observed in about 10-20% of non-small cell lung cancers (NSCLC). EGFR tyrosine kinase inhibitors (EGFR-TKIs) have been developed to target mutated EGFR. EGFR-TKIs reversibly or irreversibly bind to the ATP binding pocket of EGFR and inhibit the phosphorylation of EGFR, thereby inhibiting the activation of the EGFR signaling pathway.

Several small molecule drugs for the inhibition of activated mutant EGFR family tyrosine kinases (e.g., d746-750 mutants, L8585R mutants, and exon 20 insertion mutants) have been developed, e.g., Gefitinib, Erlotinib, Lapatinib, and others. Gefitinib or Erlotinib selectively and reversibly inhibit EGFR, and Lapatinib reversibly inhibits both EGFR and HER2, thereby arresting the growth of tumors to significantly extend the life time of the patient or to provide therapeutic advantages.

The aforementioned small-molecule inhibitors of EGFR tyrosine kinases have a common structural feature of quinazoline moiety, and tyrosine kinase inhibitors having a quinazoline moiety are disclosed in International Publication Nos. WO 99/006396, WO 99/006378, WO 97/038983, WO2000/031048, WO 98/050038, WO 99/024037, WO 2000/006555, WO 2001/098277, WO 2003/045939, WO 2003/049740 and WO 2001/012290; U.S. Pat. Nos. 7,019,012 and 6,225,318; and European Patent Nos. 0787722, 0387063, and 1292591.

It has been found that irreversible inhibitors to an EGFR target are more advantageous in overcoming the problem of resistance development, as compared to conventional reversible inhibitors. Irreversible inhibitors such as BIBW-2992 (British Journal of Cancer 98, 80, 2008), HKI-272 (Cancer Research 64, 3958, 2004) and AV-412 (Cancer Sci. 98(12), 1977, 2007) have been developed. The common feature of the aforementioned irreversible inhibitors is an acrylamide functional group at the position C-6 of a quinazoline or cyanoquinazoline residue, which forms a covalent bond with cysteine797 (Cys797, formerly called Cys773) positioned at an ATP domain of EGFR or cystein805 (Cys805) of HER2, thereby irreversibly blocking the autophosphorylation of EGFR or HER2 and efficiently inhibiting the signal transfer of cancer cells. These irreversible inhibitors exhibit higher in vitro and in vivo inhibitory activities as compared with the conventional reversible inhibitors.

International Patent Publication WO 2008/032039 discloses a novel anticancer compound having another acrylamide substituent at the position C-6 of quinazoline which shows an improved inhibition activity against EGFR tyrosine kinases.

These agents have demonstrated superior clinical efficacy with approximately 70% of patients experiencing objective responses, improved progression free survival, and quality of life compared to chemotherapy alone. However, drug resistance appeared in NSCLC patients who showed a good initial treatment response. This acquired drug resistance stems from a secondary somatic mutation at the gatekeeper position (T790M) (e.g., L8585R/T790M, d746-750/T790M mutants, exon 20 insertion/T790M mutants). About half of the patients treated with Gefitinib or Erlotinib develop resistance to Gefitinib or Erlotinib, and such drugs provide no substantial clinical effect for such EGFR T790M variant patients. The T790M mutation hinders the binding of the EGFR inhibitor to the ATP-binding site of EGFR.

Second-generation EGFR inhibitors such as afatinib, dacomitinib, poziotinib, and neratinib, while developed to overcome the acquired drug resistance, however, cause a variety of severe side effects owing to the simultaneous inhibition of wild-type EGFR. The small molecule inhibitors form a covalent bond with the cysteine residue at the position 797 (Cys797), in EGFR or cysteine805 of HER2, thereby irreversibly blocking the autophosphorylation of EGFR or HER2 and efficiently inhibiting the signal transfer of cancer cells.

Several irreversible second-generation EGFR inhibitors are described in International Publication No. WO 2008/150118, herein incorporated by reference in its entirety. The common feature of the aforementioned irreversible inhibitors is an acrylamide functional group on an aniline-quinazoline scaffold, wherein a spacer group is positioned between the acrylamide functional group and the quinazoline ring. The acrylamide functional group forms a covalent bond with the cysteine797 (Cys797) and cysteine805 (Cys805) positioned at an ATP domain of EGFR and HER2, respectively.

Third-generation EGFR inhibitors, including nazartinib, osimertinib (described in U.S. Pat. No. 8,956,235), rociletinib, HM61713 and WZ4002 exhibit characteristic specificity toward the drug-resistant T790M mutants. The common feature of the aforementioned irreversible inhibitors is an acrylamide functional group on a pyrimidine scaffold.

Approximately 10-12% of EGFR mutant NSCLC patients have an in-frame insertion within exon 20 of EGFR, and are generally resistant to EGFR-TKIs. In addition, 90% of HER2 mutations in NSCLC are exon 20 mutations. Available tyrosine kinase inhibitors of HER2 (afatinib, lapatinib, neratinib) have limited activity in EGFR/HER2 exon 20 mutant patients. Third generation EGFR TKIs (osimertinib and rociletinib) were found to have minimal activity in a patient derived xenograft model of EGFR exon 20 driven NSCLC. Similar to EGFR, activated HER2 can demonstrate a secondary mutation at the gatekeeper position (T798M), which results in resistance to tyrosine kinase inhibitors for activated HER2.

The emergence of a mutation (C797S) in EGFR and (C805S) in HER2 has resulted in new drug resistance to all known third generation EGRF-TKIs by preventing these irreversible inhibitors from forming a covalent bond with the side chain of C797 of EGFR or C805 of HER2.

### SUMMARY

One aspect of the disclosure provides an epidermal growth factor receptor (EGFR) family tyrosine kinase inhibitor comprising a functional group that can bind to the serine residue S797 in EGFR having a C797S mutation or the serine residue S805 in HER2 having a C805S mutation. T790M, T798M, and/or exon 20 insertion mutant patients treated with irreversible inhibitors can develop resistance by acquiring C797S mutation in EGFR and/or a C805S mutation in HER2. The inhibitor of the disclosure is not hindered by the T790M or T798M mutation and can advantageously bind to the serine of the C797S mutant and/or C805S mutant to block the autophosphorylation of EGFR and/or HER2 and inhibit the signal transfer of cancer cells. As used herein and unless specified otherwise, “EGFR family tyrosine kinase inhibitor” or “EGFR tyrosine kinase inhibitor” refers to a small molecule compound that inhibits an EGFR family tyrosine kinase mutant (e.g., d746-750 mutants, L8585R mutants, and/or exon 20 insertion mutants of EGFR or HER2), and secondary or tertiary mutants thereof (e.g., T790M mutants, T798M mutants C797S mutants, and C805S mutants).

As used herein and unless specified otherwise, an EGFR tyrosine kinase inhibitor is “selective” if the inhibitor does not simultaneously substantially inhibit wild-type EGFR.

As used herein and unless specified otherwise, an inhibitor can “bind to” a serine residue if the inhibitor can form a coordinate or covalent bond with a serine residue.

Another aspect of the disclosure provides an EGFR family tyrosine kinase inhibitor comprising a functional group that can bind to the serine mutant C797S EGFR and/or the C805S mutant HER2, wherein the EGFR family tyrosine kinase inhibitor comprises a compound of formula (I) or a pharmaceutically acceptable salt or solvate thereof:

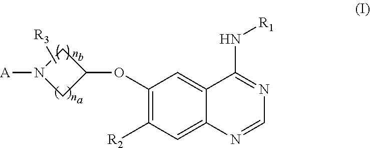

wherein,

A is:

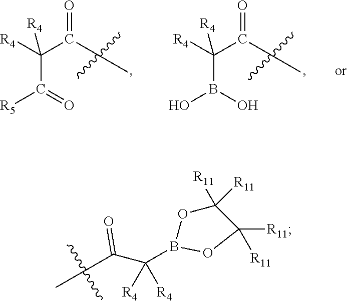

R₄ are each independently hydrogen, halogen, alkyl, cycloalkyl, perfluoroalkyl, aryl, heteroaryl, or together form cycloalkyl;

R₅ is —NHR6, —C(O)R7, alkyl, cycloalkyl, perfluoroalkyl, aryl, or heteroaryl;

R₆ is hydrogen, alkyl, cycloalkyl, perhaloalkyl, aryl, or heteroaryl; and R7 is NHR6, hydrogen, alkyl, cycloalkyl, perhaloalkyl, aryl, or heteroaryl;

R₁₁ are each independently selected from hydrogen, alkyl, alkyl-CO2R12, or can together form (═O), and R12 is selected from hydrogen or C1-6alkyl;

R₁ is a C6-10 aryl substituted with one to five X, a 5 to 10-membered heterocyclic group having at least one selected from the group consisting of N, O and S and substituted with one to five X, or C1-6 alkyl substituted with phenyl;

R₂ is hydrogen, hydroxy, C1-6 alkoxy, or C1-6 alkoxy substituted with C1-6 alkoxy or 5- or 6-membered heterocyclic group having at least one selected from the group consisting of N, O and S;

R₃ is hydrogen, —COOH, C1-6 alkyloxycarbonyl, amido N-unsubstituted, or N-substituted with Y;

na and nb are each an integer ranging from 0 to 6, with the proviso that na and nb are not simultaneously 0; and when na is 0, said

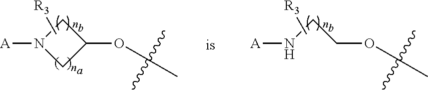

and when n_{b} is 0, said

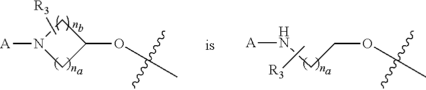

in which:

X is hydrogen, halogen, hydroxy, cyano, nitro, (mono-, di-, or trihalogeno)methyl, mercapto, C1-6 alkylthio, acrylamido, C1-6 alkyl, C2-6 alkenyl, C2-6 alkynyl, C1-6 alkoxy, aryloxy, C1-6 dialkylamino, C1-6 alkyl substituted with Z, or C1-6 alkoxy substituted with Z;

Y is hydroxy or C1-6 alkyl or C1-6 alkyl substituted with Z; and

Z is hydroxy, C1-3 alkoxy, C1-3 alkylthio, C1-3 alkylsulfonyl, di-C1-3 alkylamine, C1-6 alkyl, aryl or 5- or 6-membered aromatic or non-aromatic heterocyclic group, said heterocyclic group containing one to four of the moiety selected from the group consisting of N, O, S, SO, and SO2 and said aryl and heterocyclic group being unsubstituted, or substituted with substituents selected from the group consisting of halogen, hydroxyl, amino, nitro, cyano, C1-6 alkyl, C2-6 alkenyl, C2-6 alkynyl, C1-6 alkoxy, C1-6 monoalkylamino and C1-6 dialkylamino.

Another aspect of the disclosure provides an EGFR tyrosine kinase inhibitor comprising a functional group that can bind to the serine mutant C797S EGFR or C805S HER2, wherein the EGFR tyrosine kinase inhibitor comprises a compound of formula (II) or a pharmaceutically acceptable salt or solvate thereof:

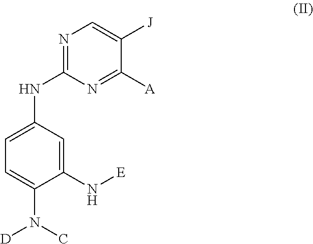

wherein,

A is:

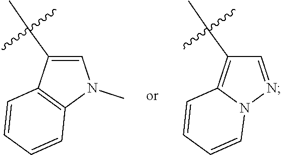 [SMILES: *c1cn(C)c2ccccc12 ; *c1cnn2ccccc12]

E is:

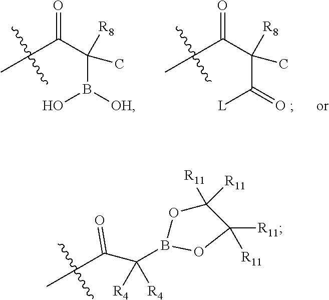

J comprises —CO₂R₁₀; halo, NHC(O)R₁₀,

R₈ are each independently selected from hydrogen, halogen, alkyl, cycloalkyl, perfluoroalkyl, aryl, heteroaryl, or together form cycloalkyl;

R₁₀ comprises hydrogen, halogen, alkyl, cycloalkyl, perfluoroalkyl, aryl, or heteroaryl;

R₁₁ are each independently selected from hydrogen, alkyl, alkyl-CO₂R₁₂, or can together form (═O), and R₁₂ is selected from hydrogen or C₁₋₆ alkyl;

C and D are each independently selected from alkyl, —N(R₈)₂, —OR₈, alkyl-W, or together can comprise a cycloalkyl;

W is selected from —N(R₈)₂ or —OR₈; and

L is selected from —CO₂NH₂, —CO₂NHR₁₀, alkyl, perfluoroalkyl, or cycloalkyl.

Another aspect of the disclosure provides an EGFR family tyrosine kinase inhibitor comprising a functional group that can bind to the serine mutant C797S EGFR and/or C805S HER2, wherein the EGFR family tyrosine kinase inhibitor comprises a compound of formula (III) or a pharmaceutically acceptable salt or solvate thereof:

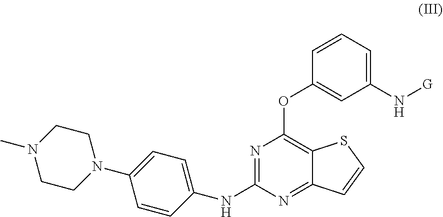

wherein

G is:

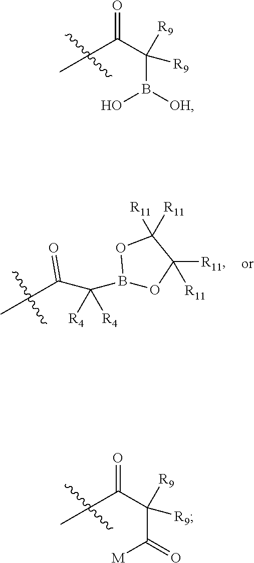

R₉ are each independently selected from hydrogen, halogen, alkyl, cycloalkyl, perfluoroalkyl, aryl, heteroaryl, or together form cycloalkyl;

M is selected from —CO₂NH₂, —CO₂NHR₁₀, alkyl, perfluoroalkyl, or cycloalkyl, optionally comprising alkyl branches on one or more carbon atoms;

R₁₀ comprises hydrogen, halogen, alkyl, cycloalkyl, perfluoroalkyl, aryl, or heteroaryl; and

R₁₁ are each independently selected from hydrogen, alkyl, alkyl-CO₂R₁₂, or can together form (═O), and R₁₂ is selected from hydrogen or C₁₋₆ alkyl.

Another aspect of the disclosure provides a pharmaceutical composition comprising an EGFR tyrosine kinase inhibitor comprising a functional group that can bind to the serine mutant (C797S) of EGFR and/or (C805S) of HER2, or a pharmaceutically acceptable salt or solvate thereof as an active ingredient and a pharmaceutically acceptable carrier.

Another aspect of the disclosure provides a method of treating a subject having an EGFR C797S mutation or a HER2 C805S mutation comprising administering to the subject a pharmaceutically effective amount of an EGFR family tyrosine kinase inhibitor compound or its pharmaceutically acceptable salt or solvate according to the disclosure.

For the compounds and compositions described herein, optional features are contemplated to be selected from the various aspects, embodiments, and examples provided herein.

Further aspects and advantages will be apparent to those of ordinary skill in the art from a review of the following detailed description. While the compounds and compositions are susceptible of embodiments in various forms, the description hereafter includes specific embodiments with the understanding that the disclosure is illustrative and is not intended to limit the invention to the specific embodiments described herein.

### DETAILED DESCRIPTION

The disclosure provides an epidermal growth factor receptor (EGFR) family tyrosine kinase inhibitor comprising a functional group that can bind to the serine residue S797 in EGFR having a C797S mutation and/or the serine residue S805 in HER2 having a C805S mutation. Advantageously, the EGFR tyrosine kinase inhibitor comprising a functional group that can bind to the serine in the C797S mutant of EGFR and/or C805S mutant of HER2 also selectively inhibits the mutant T790M/C797S EGFR and/or the mutant T798M/C805 HER2, provided the C797S and/or C805S mutation coexists with the T790M mutation or T798M mutation, respectively. Without intending to be bound by theory, it is believed that the mutation of EGFR involves the replacement of cysteine 797 with serine and the mutation of HER2 involves the replacement of cysteine 805 with serine, and the nucleophilic hydroxyl group of serine can bind with an electron deficient functional group on an EGFR tyrosine kinase inhibitor, such as a boronic acid or an electron deficient carbonyl. Electron deficient carbonyls can be used as a serine trap such that the proliferation signaling of the protein is disrupted through a bond formation.

The EGFR family tyrosine kinase inhibitor comprising a functional group that can bind to the serine in the C797S mutant of EGFR and/or the C805S mutant of HER2 of the disclosure can comprise a compound of formula (I) or a pharmaceutically acceptable salt or solvate

thereof:

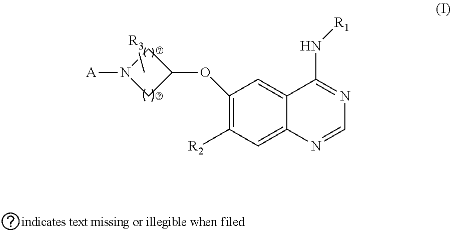

wherein,

A is:

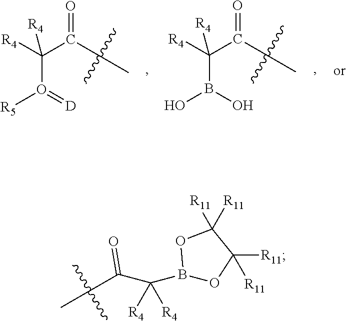

R₄ are each independently hydrogen, halogen, alkyl, cycloalkyl, perfluoroalkyl, aryl, heteroaryl, or together form cycloalkyl;

R₅ is —NHR₆, —C(O)R₇, alkyl, cycloalkyl, perfluoroalkyl, aryl, or heteroaryl;

R₆ is hydrogen, alkyl, cycloalkyl, perhaloalkyl, aryl, or heteroaryl; and R₇ is NHR₆, hydrogen, alkyl, cycloalkyl, perhaloalkyl, aryl, or heteroaryl;

R₁₁ are each independently selected from hydrogen, alkyl, alkyl-CO₂R₁₂, or can together form (═O), and R₁₂ is selected from hydrogen or C₁₋₆ alkyl;

R₁ is a C₆₋₁₀ aryl substituted with one to five X, a 5 to 10-membered heterocyclic group having at least one selected from the group consisting of N, O and S and substituted with one to five X, or C₁₋₆ alkyl substituted with phenyl;

R₂ is hydrogen, hydroxy, C₁₋₆ alkoxy, or C₁₋₆ alkoxy substituted with C₁₋₆ alkoxy or 5- or 6-membered heterocyclic group having at least one selected from the group consisting of N, O and S;

R₃ is hydrogen, —COOH, C₁₋₆ alkyloxycarbonyl, amido N-unsubstituted or amido N-substituted with Y;

nₐ and n_{b} are each an integer ranging from 0 to 6, with the proviso that nₐ and n_{b} are not simultaneously 0; and when nₐ is 0, said

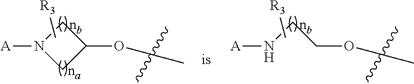

and when n_{b} is 0, said

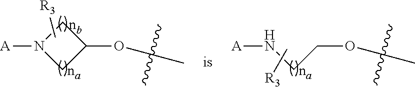

in which:

X is hydrogen, halogen, hydroxy, cyano, nitro, (mono-, di-, or trihalogeno)methyl, mercapto, C₁₋₆ alkylthio, acrylamido, C₁₋₆ alkyl, C₂₋₆ alkenyl, C₂₋₆ alkynyl, C₁₋₆ alkoxy, aryloxy, C₁₋₆ dialkylamino, C₁₋₆ alkyl substituted with Z or C₁₋₆ alkoxy substituted with Z;

Y is hydroxy or C₁₋₆ alkyl or C₁₋₆ alkyl substituted with Z; and

Z is hydroxy, C₁₋₃ alkoxy, C₁₋₃ alkylthio, C₁₋₃ alkylsulfonyl, alkylamine, C₁₋₆ alkyl, aryl or 5- or 6-membered aromatic or non-aromatic heterocyclic group, said heterocyclic group containing one to four of the moiety selected from the group consisting of N, O, S, SO, and SO2 and said aryl and heterocyclic group being unsubstituted, or substituted with substituents selected from the group consisting of halogen, hydroxyl, amino, nitro, cyano, C₁₋₆ alkyl, C₂₋₆ alkenyl, C₂₋₆ alkynyl, C₁₋₆ alkoxy, C₁₋₆ monoalkylamino and C₁₋₆ dialkylamino.

The term “halogen” refers to fluoro, chloro, bromo, or iodo, unless otherwise indicated. In embodiments, each halogen can be individually selected from the group consisting of fluoro, chloro, bromo, or iodo. In embodiments, at least one halogen comprises fluoro. In embodiments, all halogens comprise fluoro. In embodiments, at least one halogen comprises chloro.

The term “alkyl” refers to saturated monovalent hydrocarbon radicals having straight, cyclic, or branched moieties (i.e., can be unsubstituted or substituted), unless otherwise indicated. In embodiments, each alkyl can be individually selected from the group consisting of unsubstituted alkyl and alkyl substituted with methyl, ethyl, propyl, or a combination thereof.

In embodiments wherein X is aryloxy, X can be phenyloxy. In embodiments wherein Y is C₁₋₆ alkyl or C₁₋₆ alkyl substituted with Z, the C₁₋₆ alkyl can comprise one to four moieties selected from the group consisting of N, O, S, SO, and SO2. In embodiments wherein Z is aryl, Z can be phenyl. In embodiments wherein Z is aryl, said aryl group can be a C₅₋₁₂ monocyclic or bicyclic aromatic or non-aromatic group containing one to four of moieties selected from the group consisting of N, O, S, SO, and SO₂.

In embodiments, R₆ is C₁₋₆ alkyl or C₃₋₇ cycloalkyl. In embodiments, R₇ is C₁₋₆ alkyl or C₃₋₇ cycloalkyl. In embodiments, R₁ is a C₆ aryl substituted with 3 X. In embodiments, nₐ and n_{b} are both 2. In embodiments, R₂ is methoxy. In embodiments, R₃ is hydrogen.

In embodiments, A is

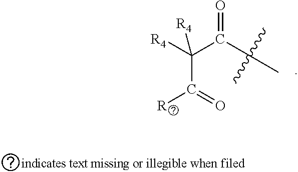

In embodiments A is

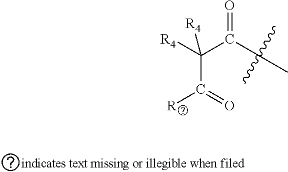

and R₄ are both halogen. In embodiments, A is

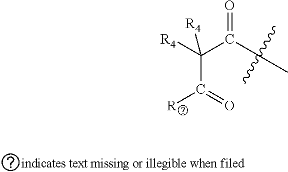

and R₄ are both fluoro.

In embodiments, A is

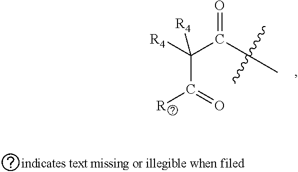

R₅ is —C(O)R₇, R₇ is C₁₋₆ alkyl or C₃₋₇ cycloalkyl, R₁ is a C₆ aryl substituted with 3 X, nₐ and n_{b} are both 2, R₂ is methoxy, R₃ is hydrogen, and R₄ are each individually halogen.

In embodiments, A is

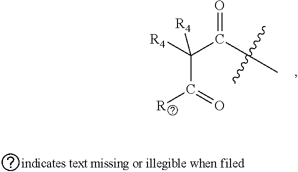

R₅ comprises C(O)R₇, and R₇ comprises NHR₆. In embodiments, A is

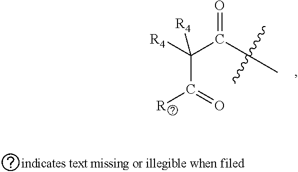

R₅ comprises C(O)R₇, R₇ comprises NHR₆, and R₄ are each individually selected from fluoro, C₁₋₆ alkyl, C₃₋₇ cycloalkyl, or together form cycloalkyl. In embodiments, A is

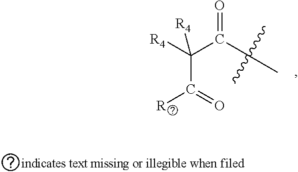

R₅ comprises C(O)R₇, R₇ comprises NHR6, and R₆ is selected from hydrogen, C1-6 alkyl, C3-7 cycloalkyl, perhaloalkyl, aryl, and heteroaryl.

In embodiments, A is

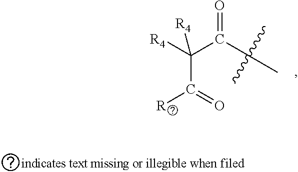

R₅ comprises C(O)R₇, R₇ comprises NHR₆, C₁₋₆ alkyl or C₃₋₇ cycloalkyl, R₆ is selected from hydrogen, C₁₋₆ alkyl, C₃₋₇ cycloalkyl, perhaloalkyl, aryl, and heteroaryl, R₄ are each individually selected from fluoro, C₁₋₆ alkyl, C₃₋₇ cycloalkyl, or together form cycloalkyl, R₁ is a C₆ aryl substituted with 3 X, nₐ and n_{b} are both 2, R₂ is methoxy, and R₃ is hydrogen.

In embodiments, A is

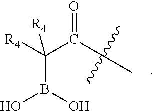

In embodiments, A is

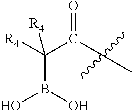

and R₄ are each independently hydrogen, halogen, C₁₋₆ alkyl, C₃₋₇ cycloalkyl, perfluoroalkyl, cycloalkyl, aryl, heteroaryl, or together form C₃₋₇ cycloalkyl. In embodiments, A is

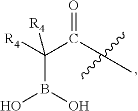

R₄ are each independently hydrogen, halogen, C₁₋₆ alkyl, C₃₋₇ cycloalkyl, perfluoroalkyl, cycloalkyl, aryl, heteroaryl, or together form C₃₋₇ cycloalkyl, R₁ is a C₆ aryl substituted with 3 X, nₐ and n_{b} are both 2, R₂ is methoxy and R₃ is hydrogen.

In embodiments, A is

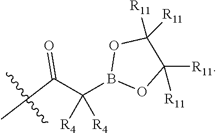

In embodiments,

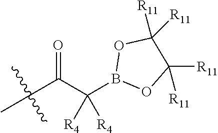

### A is

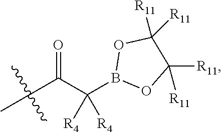

wherein two R₁₁ together comprise (═O)
and two R₁₁ each comprise methyl-CO₂R₁₂, wherein R₁₂ Is hydrogen, such that A is

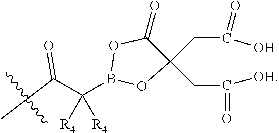

In embodiments, A is

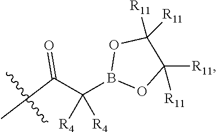

for example

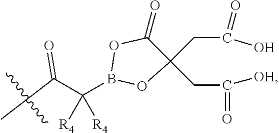

and R₄ are each independently hydrogen, halogen, C₁₋₆ alkyl, C₃₋₇ cycloalkyl, perfluoroalkyl, cycloalkyl, aryl, heteroaryl, or together form C₃₋₇ cycloalkyl.

In embodiments, A is

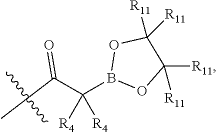

for example,

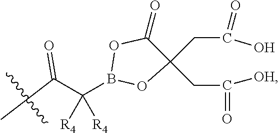

R₄ are each independently hydrogen, halogen, C₁₋₆ alkyl, C₃₋₇ cycloalkyl, perfluoroalkyl, cycloalkyl, aryl, heteroaryl, or together form C₃₋₇ cycloalkyl, R₁ is a C₆ aryl substituted with 3 X, nₐ and n_{b} are both 2, R₂ is methoxy and R₃ is hydrogen.

Examples of compounds of formula (I) according to the disclosure include:
1) 4-(4-((4-(3,4-dichloro-2fluorophenyl)amino)-7-methoxyquinazolin-6-yl)oxy)piperidin-1-yl)-N,3,3-trimethyl-2,4-dioxobutanamide;
2) (2-(4-((4-((3,4-dichloro-2-fluorophenyl)amino)-7-methoxyquinazolin-6-yl)oxy)piperidin-1-yl)-2-oxoethyl)boronic acid; and
3) 1-(4-((4-((3,4-dichloro-2-fluorophenyl)amino)-7-methoxyquinazolin-6-yl)oxy)piperidin-1-yl)-2,2-difluorobutane-1,3-dione.

Another aspect of the disclosure provides an EGFR family tyrosine kinase inhibitor comprising a functional group that can bind to the serine in C797S of EGFR and/or the serine in C805S of HER2, wherein the EGFR family inhibitor comprises a compound of formula (II) or a pharmaceutically acceptable salt or solvate thereof:

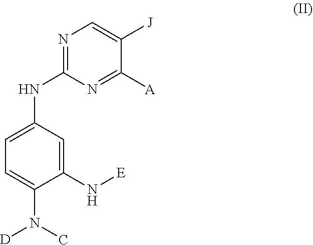

wherein,

A is:

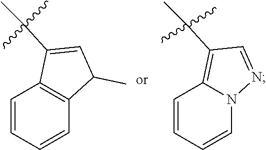 [SMILES: *C1=CC(C)c2ccccc21 ; *c1cnn2ccccc12]

E is:

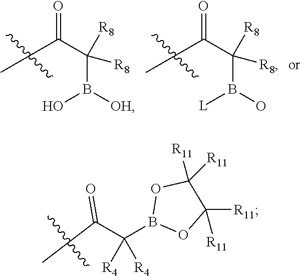

J comprises —CO₂R₁₀, halo, —NHC(O)R₁₀;

R₈ are each independently selected from hydrogen, halogen, alkyl, cycloalkyl, perfluoroalkyl, aryl, heteroaryl, or together form cycloalkyl;

R₁₀ comprises hydrogen, halogen, alkyl, cycloalkyl, perfluoroalkyl, aryl, or heteroaryl;

R₁₁ are each independently selected from hydrogen, alkyl, alkyl-CO₂R₁₂, or can together form (═O), and Rig is selected from hydrogen or C₁₋₆ alkyl;

C and D are each independently selected from alkyl, —N(R₈)₂, —OR₈, alkyl-W, or together can comprise a cycloalkyl;

W is selected from —N(R₈)₂ or —OR₈; and

L is selected from —CO₂NH₂, —CO₂NHR₁₀, alkyl, perfluoroalkyl, or cycloalkyl.

In embodiments, J comprises halo. In embodiments, J comprises chloro. In embodiments, J comprises —NHC(O)R₁₀ and R₁₀ comprises a C₁₋₆ alkyl or C₃₋₇ cycloalkyl, optionally substituted C₁₋₆ alkyl or C₃₋₇ cycloalkyl. In embodiments, J comprises —CO₂R₁₀ and R₁₀ comprises a C₁₋₆ alkyl or C₃₋₇ cycloalkyl, optionally substituted C₁₋₆ alkyl or C₃₋₇ cycloalkyl. In embodiments, J comprises —CO₂R₁₀ and R₁₀ comprises tert-butyl or cyclohexyl, or J-NHC(O)R₁₀ and R₁₀ comprises iso-propyl.

In embodiments, one or both of C and D are substituted with C₁₋₃ alkyl on one or more carbon atoms. In embodiments, L is C₁₋₈ alkyl or C₃₋₇ cycloalkyl and are unsubstituted or substituted with C₁₋₃ alkyl on one or more carbon atoms. In embodiments, R₈ are each independently selected from C₁₋₆ alkyl, C₃₋₇ cycloalkyl, or together form C₃₋₇ cycloalkyl. In embodiments, one or both R₈ are substituted with C₁₋₃ alkyl on one or more carbon atoms.

In embodiments, E is

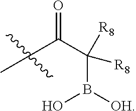

In embodiments, E is

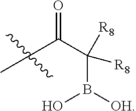

one or both of C and D are substituted with C₁₋₃ alkyl on one or more carbon atoms, and R₈ are each independently selected from C₁₋₆ alkyl, C₃₋₇ cycloalkyl, or together form C₃₋₇ cycloalkyl.

In embodiments, E is

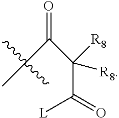

In embodiments, E is

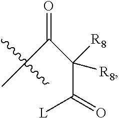

one or both of C and D are substituted with C₁₋₃ alkyl on one or more carbon atoms, L is C₁₋₈ alkyl, C₁₋₈ perfluoroalkyl, or C₃₋₇ cycloalkyl and are unsubstituted or substituted with C₁₋₃ alkyl on one or more carbon atoms, and R₈ are each independently selected from C₁₋₆ alkyl, C₃₋₇ cycloalkyl, or together form C₃₋₇ cycloalkyl.

In embodiments, E is

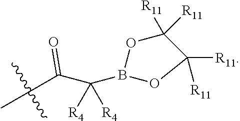

In embodiments, E is

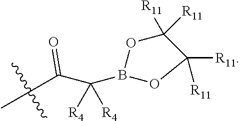

wherein two R₁₁ together comprise (═O) and two R₁₁ comprise methyl-CO₂R₁₂, wherein R₁₂ is hydrogen, such that E is

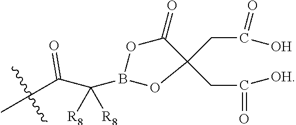

In embodiments, E is

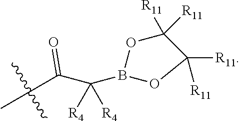

for example,

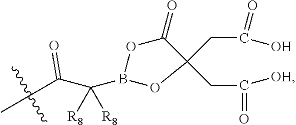

one or both of C and D are substituted with C₁₋₃ alkyl on one or more carbon atoms, and R₈ are each independently selected from C₁₋₆ alkyl, C₃₋₇ cycloalkyl, or together form C₃₋₇ cycloalkyl.

Examples of compounds of formula (II) according to the disclosure include:
1) 2-((2-((2-(dimethylamino)ethyl)(methyl)amino)-5-((4-(1-methyl-1H-indol yl)pyrimidin-2-yl)amino)phenyl)amino)-2-oxoethyl)boronic acid; and
2) N-(2-((2-(dimethylamino)ethyl)(methyl)amino)-5-((4-1-methyl-1H-indol yl)pyrimidin-2-yl)amino)phenyl)-2,2-difluoro-3-oxobutanamide.

Another aspect of the disclosure provides an EGFR family tyrosine kinase inhibitor comprising a functional group that can bind to the serine residue in the C797S mutant of EGFR and/or the C805S mutant of HER2, wherein the EGFR family tyrosine kinase inhibitor comprises a compound of formula (III) or a pharmaceutically acceptable salt or solvate thereof:

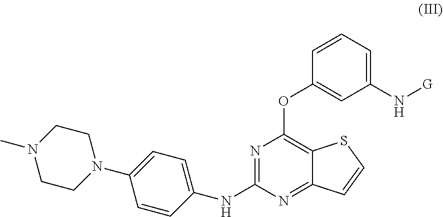

wherein

G is:

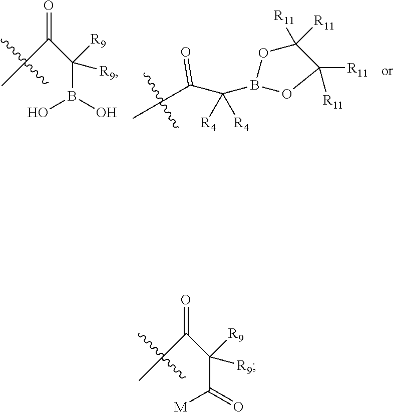

R₉ are each independently selected from hydrogen, halogen, alkyl, cycloalkyl, perfluoroalkyl, aryl, heteroaryl, or together form cycloalkyl;

M is selected from —CO₂NH₂, —CO₂NHR₁₀, alkyl, perfluoroalkyl, or cycloalkyl, optionally comprising alkyl branches on one or more carbon atoms;

R₁₀ comprises hydrogen, halogen, alkyl, cycloalkyl, perfluoroalkyl, aryl, or heteroaryl;

and

R₁₁ are each independently selected from hydrogen, alkyl, alkyl-CO₂R₁₂, or can together form (═O), and R₁₂ is selected from hydrogen or C₁₋₆ alkyl.

In embodiments, R₉ are each independently selected from C₁₋₆ alkyl, C₃₋₇ cycloalkyl, or together form a C₃₋₇ cycloalkyl. In embodiments, one or both R₉ are substituted with C₁₋₃ alkyl on one or more carbon atoms. In embodiments, M is C₁₋₈ alkyl, C₁₋₈ perfluoroalkyl, or C₃₋₇ cycloalkyl and M is unsubstituted or substituted with C₁₋₃ alkyl on one or more carbon atoms.

In embodiments, R₉ are each independently selected from C₁₋₆ alkyl, C₃₋₇ cycloalkyl, or together form a C₃₋₇ cycloalkyl and each R₉ is either unsubstituted or substituted with C₁₋₃ alkyl on one or more carbon atoms, M is C₁₋₈ alkyl or C₃₋₇ cycloalkyl, and M is unsubstituted or substituted with C₁₋₃ alkyl on one or more carbon atoms.

In embodiments, G is

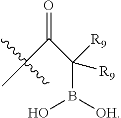

In embodiments, G is

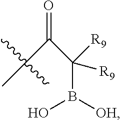

R₉ are each independently selected from hydrogen, C₁₋₆ alkyl, C₃₋₇ cycloalkyl, or together form a C₃₋₇ cycloalkyl, and each R₉ is either unsubstituted or substituted with C₁₋₃ alkyl on one or more carbon atoms.

In embodiments, G is

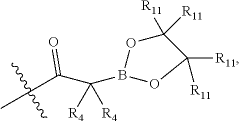

In embodiments, G is

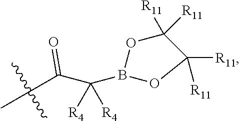

wherein two R₁₁ together comprise (═O) and two R₁₁ comprise methyl-CO₂R₁₂, wherein R₁₂ is hydrogen such that G is

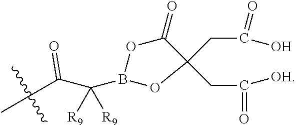

In embodiments, G is

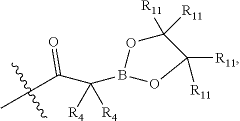

for example,

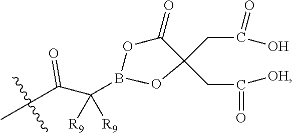

R₉ are each independently selected from hydrogen, C₁₋₆ alkyl, C₃₋₇ cycloalkyl, or together form a C₃₋₇ cycloalkyl, and each R₉ is either unsubstituted or substituted with C₁₋₃ alkyl on one or more carbon atoms.

In embodiments, G is

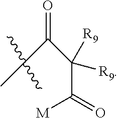

In embodiments, G is

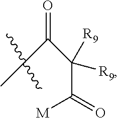

R₉ are each independently selected from C₁₋₆ alkyl, C₃₋₇ cycloalkyl, or together form a C₃₋₇ cycloalkyl and each R₉ is either unsubstituted or substituted with C₁₋₃ alkyl on one or more carbon atoms, M is C₁₋₈ alkyl or C₃₋₇ cycloalkyl, and M is unsubstituted or substituted with C₁₋₃ alkyl on one or more carbon atoms.

A compound of formula (I) of the present disclosure may be prepared, for example, by the procedure shown in Reaction Scheme (I) (see [Bioorg. Med. Chem. Lett., 2001; 11: 1911] and International Patent Publication WO 2003/082831):

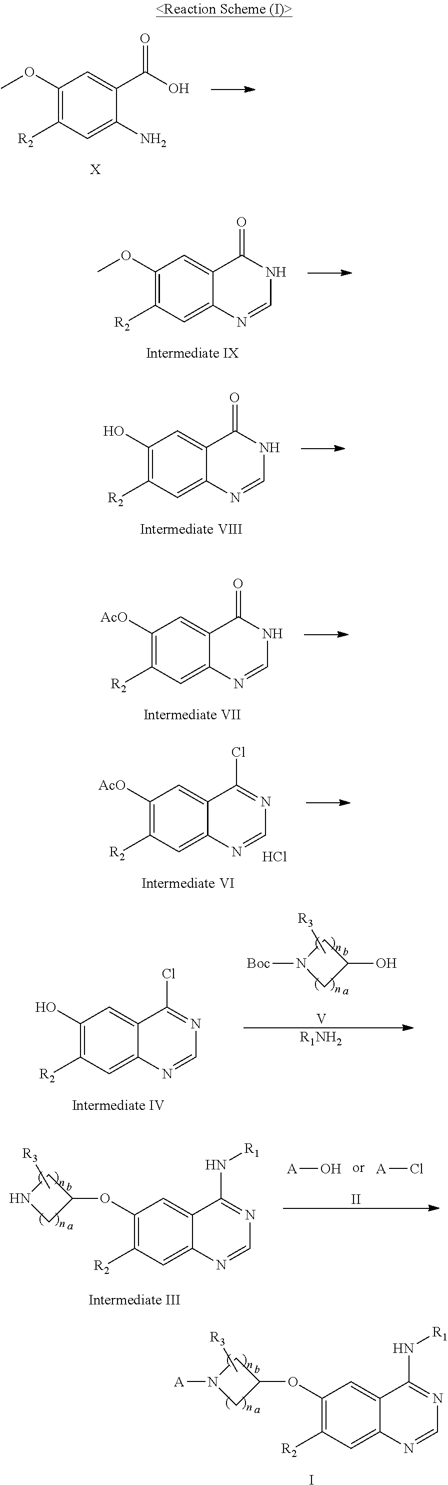

wherein,

A, R₁, R₂, R₃, nₐ and n_{b} have the same meanings as defined above for compounds of formula (I).

In Reaction Scheme (I), a compound of formula (X) is subjected to a condensation reaction with formamidine hydrochloride at a high temperature (e.g. 210° C.) to form a compound of intermediate formula (IX), followed by a reaction with L-methionine in an organic acid (e.g., methanesulfonic acid), inducing the removal of methyl at the position C-6 of the compound of intermediate formula (IX), to form a compound of intermediate formula (VIII).

Subsequently, the compound of intermediate formula (VIII) is subjected to a protection reaction in a base (e.g., pyridine) and an anhydrous acetic acid to form a compound of intermediate formula (VII), followed by a reaction with an inorganic acid (e.g., thionylchloride or phosphorous oxychloride) in the presence of a catalytic amount of dimethylformamide under a reflux condition, to form a compound of intermediate formula (VI) in a form of hydrochlorate.

The compound of intermediate formula (VI) is added to an ammonia-containing alcohol solution (e.g., a 7N ammonia-containing methanol solution), which was stirred, inducing the removal of acetyl therefrom, to form a compound of intermediate formula (IV). The compound of intermediate formula (IV) is subjected sequentially to Mitsunobu reaction with a compound of formula (V) and a substitution reaction with R1NH2 in an organic solvent (e.g., 2-propanol or acetonitrile) to introduce R1 thereto. The resulting compound is subjected to a reaction with an organic or inorganic acid (e.g., trifluoroacetic acid or heavy hydrochloric acid) in an organic solvent (e.g., methylene chloride), inducing the removal of t-butoxycarbonyl, to form a compound of intermediate formula (II). In the Mitsunobu reaction, diisopropyl azodicarboxylate, diethyl azodicarboxylate, di-t-butyl azodicarboxylate or triphenylphosphine may be employed.

Subsequently, a compound of intermediate formula (I) of the present disclosure is prepared by subjecting the compound of intermediate formula (II) to a condensation reaction with a compound of intermediate formula (III), A-Cl, in a mixture of an organic solvent (e.g., tetrahydrofuran and water or methylene chloride in the presence of an inorganic or organic base (e.g., sodium bicarbonate, pyridine or triethylamine); or by subjecting the compound of intermediate formula (II) to a condensation reaction with a compound of intermediate formula (III), A-OH, in an organic solvent (e.g., tetrahydrofuran or methylene chloride) in the presence of a coupling agent (e.g., 1-ethyl-3-(3-dimethylaminopropyl)-carbodiimide (EDC) or 2-(1H-7-azabenzotriazol-1-yl)-1,1,3,3-tetramethyl europium hexafluorophosphate methanaminium (HATU)).

A compound of formula (II) of the present disclosure may be prepared, for example, by the procedure shown in Reaction Scheme (IIA and IIB) (See J. Med. Chem., 2014, 57 (20), pp 8249-8267).

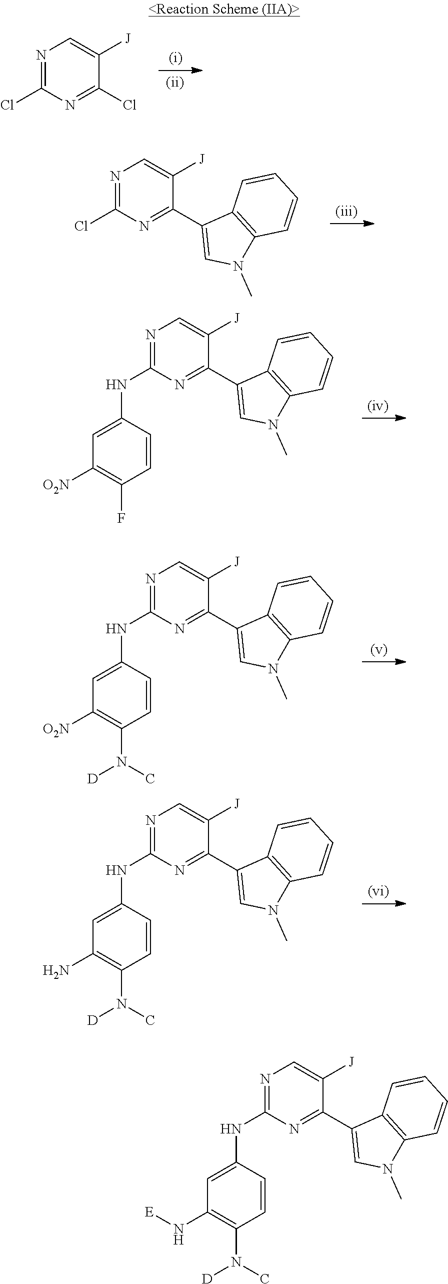

wherein J, D, and C have the same meanings as defined above for compounds of formula (II).

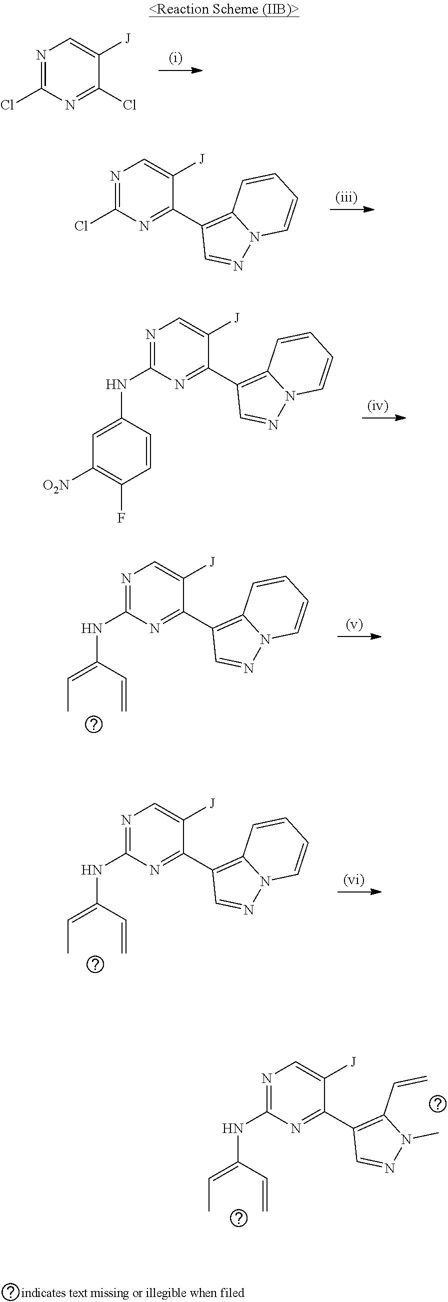

wherein J, D, and C have the same meanings as defined above for compounds of formula (II).

The difference between Reaction Scheme (IIA) and Reaction Scheme (IIB) is the addition of indole (IIA) or pyrazolo[1,5-a]pyridine (IIB) in step (i). In Reaction Scheme (II), 2,4-dichloropyrimidine substituted with group J is reacted with MeMgBr (1 eq, 3.2 M in 2-methyl THF) and 1 eq of indole or pyrazolo[1,5-a]pyridine in THF at 0° C. and warmed to 60° C. (i). 1.05 eq sodium hydroxide and 1.05 eq methyl iodide are added at 0° C. in Reaction Scheme (IIA) to replace the hydrogen on the indole nitrogen with a methyl group (ii). 4-fluoro-5-nitroaniline (1.05 eq), tosic acid (1.1 eq) and 2-pentanol were added to the mixture at 125° C. (iii). Subsequently, 2.2 eq of the N(D)(C) moiety in DMA was added at 140° C. (iv), followed by 3 eq of iron, 0.7 eq of ammonium chloride, ethanol and water at 100° C. (v). Subsequently E-Cl or E-OH (1 M, THF, 1 eq) in DIPEA and THF at 0° C. was added to incorporate the functional group that can bind to serine, thereby forming a compound of formula (II).

A compound of formula (III) of the present disclosure may be prepared, for example, by the procedure shown in Reaction Scheme (III) (See International Patent Application Publication No. WO 2011/162515A2).

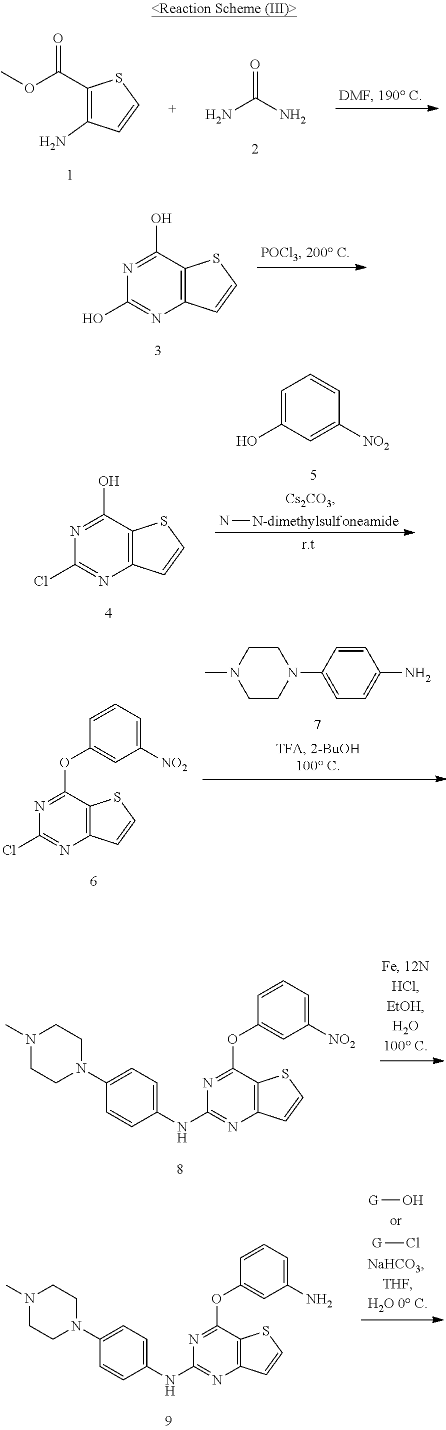

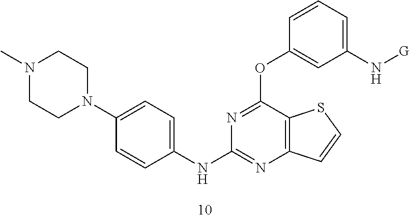

wherein G has the same meaning as defined above for compounds of formula (III).

In Reaction Scheme (III), a compound of formula 1 is subjected to a condensation reaction with urea in an organic solvent (e.g., N,N-dimethylformamide, N,N-dimethylacetamide, or N-methyl pyrrolidone) at a temperature ranging from reflux temperature to 200° C., or with potassium cyanate under an acidic condition such as 6% to 50% of aqueous acetic acid at a temperature ranging from room temperature to 100° C., to obtain a condensed compound of formula 2.

The compound of formula 3 thus obtained is refluxed with stirring in the presence of a chlorinating agent (e.g., phosphorus oxychloride or thionyl chloride) to obtain a chlorinated compound of formula 4, followed by a reaction in an organic solvent (e.g., dimethylsulfoxide, N,N-dimethylformamide, N,N-dimethylacetamide, N-methyl pyrrolidone, acetonitrile, tetrahydrofuran, 1,4-dioxane, toluene or benzene) in the presence of an inorganic base (e.g., cesium carbonate, sodium carbonate or potassium carbonate) at a temperature ranging from room temperature to 100° C., inducing substitution at the C-4 position of the compound of formula 4 with the compound of formula 5, to obtain a compound of formula 6.

The compound of formula 6 is reacted with a compound of formula 7 in an alcohol solution (e.g., 2-propanol or 2-butanol) in the presence of an organic acid (e.g., trifluoroacetic acid (TFA)) at a temperature ranging from 70° C. to reflux temperature to obtain a compound of formula 8.

The compound of formula 8 is subjected to a hydrogenation using a palladium/carbon catalyst, or a reduction reaction mediated with Fe, to obtain an aniline compound of formula 9. Subsequently, the aniline compound of formula 9 is subjected to a reaction with a chloride of group G in an organic solvent (e.g., methylene chloride or tetrahydrofuran) or a mixed solvent such as 50% aqueous tetrahydrofuran in the presence of an inorganic base (e.g., sodium bicarbonate) or organic base (e.g., triethylamine or diisopropylethylamine) at a low temperature ranging from −10° C. to 10° C.; or with an acid of group G in pyridine using a coupling agent (e.g., 1-ethyl-3-(3-dimethylaminopropyl)carbodiimide (EDCl) or 2-(1H-7-azabenzotriazol-1-yl)-1,1,3,3-tetramethyl uranium hexafluoro phosphate methaneaminium (HATU)), to obtain a compound of formula 10, corresponding to an EGFR family tyrosine kinase inhibitor compound of formula (III).

A compound of formula (I)-(III) of the present disclosure can also be used in the form of a pharmaceutically acceptable salt or solvate formed with an inorganic or organic acid such as hydrochloric acid, hydrobromic acid, sulfuric acid, phosphoric acid, nitric acid, acetic acid, glycolic acid, lactic acid, pyruvic acid, malonic acid, succinic acid, glutaric acid, fumaric acid, malic acid, mandelic acid, tartaric acid, citric acid, ascorbic acid, palmitic acid, maleic acid, hydroxymaleic acid, benzoic acid, hydroxybenzoic acid, phenylacetic acid, cinnamic acid, salicylic acid, methanesulfonic acid, benzenesulfonic acid and toluenesulfonic acid.

The compounds of the disclosure or a pharmaceutically acceptable salt or solvate thereof selectively and efficiently inhibit the growth of cancer cells induced by activated epidermal growth factor family tyrosine kinase with a cysteine to serine mutation, and provide enhanced anticancer effects when combined with another anticancer agent. Namely, the compounds of the disclosure or a pharmaceutically acceptable salt or solvate thereof are useful for enhancing the effects of an anticancer agent selected from the group consisting of cell signal transduction inhibitors, mitosis inhibitors, alkylating agents, antimetabolites, antibiotics, growth factor inhibitors, cell cycle inhibitors, topoisomerase inhibitors, biological reaction modifiers, antihormonal agents and antiandrogen.

Therefore, the present disclosure provides a pharmaceutical composition for inhibiting cancer cell growth comprising one or more of the compound of formula (I), formula (II), formula (III), a pharmaceutically acceptable salt or solvate of the foregoing, or a combination of the foregoing as an active ingredient and a method of treating a subject having an EGFR C797S mutation and/or an HER2 C805S mutation comprising administering to the subject a pharmaceutically effective amount of an EGFR family tyrosine kinase inhibitor compound or its pharmaceutically acceptable salt or solvate according to the disclosure.

The compounds of the disclosure or a pharmaceutically acceptable salt or solvate thereof may be administered orally or parenterally as an active ingredient in an effective amount ranging from about 0.01 to 100 mg/kg, preferably 0.2 to 50 mg/kg body weight per day in case of mammals including human in a single dose or in divided doses. The dosage of the active ingredient may be adjusted in light of various relevant factors such as the condition of the subject to be treated, type and seriousness of illness, administration rate, and opinion of doctor. In certain cases, an amount less than the above dosage may be suitable. An amount greater than the above dosage may be used unless it causes deleterious side effects and such amount can be administered in divided doses per day.

The pharmaceutical composition may be formulated in accordance with any of the conventional methods in the form of tablet, granule, powder, capsule, syrup, emulsion or microemulsion for oral administration, or for parenteral administration including intramuscular, intravenous and subcutaneous routes.

The pharmaceutical composition for oral administration may be prepared by mixing the active ingredient with a carrier such as cellulose, calcium silicate, corn starch, lactose, sucrose, dextrose, calcium phosphate, stearic acid, magnesium stearate, calcium stearate, gelatin, talc, surfactant, suspension agent, emulsifier and diluent. Examples of the carrier employed in the injectable composition of the present disclosure are water, a saline solution, a glucose solution, a glucose-like solution, alcohol, glycol ether (e.g., polyethylene glycol 400), oil, fatty acid, fatty acid ester, glyceride, a surfactant, a suspension agent and an emulsifier.

The following Examples are intended to further illustrate the present disclosure without limiting its scope.

### EXAMPLES

### Example 1: Preparation of a Compound of Intermediate Formula III

### (1-1) 6,7-dimethoxyquinazolin-4(3H)-one

36.9 g of 4,5-dimethoxyanthranilic acid was mixed with 25.0 g of formamidine hydrochloride, and the mixture was stirred at 210° C. for 30 minutes. After completion of the reaction, the solid thus obtained was cooled to room temperature, stirred with 200 ml (0.33 M) of aqueous sodium hydroxide and filtered under a reduced pressure. The solid thus obtained was washed with water and air-dried to obtain the title compound (24.6 g, 64%).

¹H-NMR (300 MHz, DMSO-d₆) δ 7.99 (s, 1H), 7.44 (s, 1H), 7.13 (s, 1H), 3.90 (s, 3H), 3.87 (s, 3H).

### (1-2) 6-hydroxy-7-methoxyquinazolin-4(3H)-one

3.06 g of the compound obtained in (1-1) was diluted with 20 ml of methanesulfonic acid. 2.66 g of L-methionine was added to the resulting solution and stirred at 100° C. for 22 hours. Ice was added to the reaction mixture and neutralized with 40% aqueous sodium hydroxide to induce the crystallization of the product. The solid was filtered under a reduced pressure, washed with water, and air-dried to obtain the title compound (2.67 g, 94%).

¹H-NMR (300 MHz, DMSO-d₆) δ 11.94 (s, 1H), 9.81 (s, 1H), 7.92 (s, 1H), 7.39 (s, 1H), 7.11 (s, 1H), 3.91 (s, 3H).

### (1-3) 7-methoxy-4-oxo-3,4-dihydroquinazolin-6-yl acetate

6.08 g of the compound obtained in (1-2) was dissolved in a mixture of 550 ml of acetic acid and 7 ml of pyridine, and the resulting solution was stirred 100° C. for 3 hours. The reaction solution was cooled to room temperature, and ice was added thereto to induce the crystallization of the product. The solid was filtered under a reduced pressure, washed with water, and air-dried to obtain the title compound (4.87 g, 65%).

¹H-NMR (300 MHz, DMSO-d₆) δ 12.21 (s, 1H), 8.09 (s, 1H), 7.76 (s, 1H), 7.28 (s, 1H), 3.91 (s, 3H), 2.30 (s, 3H).

### (1-4) 4-chloro-7-methoxyquinazolin-6-yl acetate hydrochloride salt

4.87 g of the compound obtained in (1-3) was dissolved in a mixture of 33 ml thionylchloride and 6 ml of phosphorus oxychloride. Two drops of dimethylformamide were added to the resulting solution and stirred at 120° C. for 7 hours. The reaction solution was cooled to room temperature and the solvent was removed therefrom under a reduced pressure, to obtain a residue. Toluene was added the residue, and the resulting solution was concentrated under a reduced pressure to remove the solvent, and this procedure was repeated 2 more times. The solid thus obtained was dried under a reduced pressure to obtain the title compound (5.16 g).

¹H-NMR (300 MHz, DMSO-d₆) δ 9.01 (s, 1H), 8.02 (s, 1H), 7.64 (s, 1H), 4.02 (s, 3H), 2.35 (s, 3H).

### (1-5) 4-chloro-7-methoxyquinazolin-6-ol

2 g of the compound obtained in (1-4) was added to 25 ml of 7 N ammonia methanol solution. The mixture was stirred at room temperature for 1 hour, the solid formed in the reacting mixture was filtered, washed with diethylether, and dried to obtain the title compound (1.43 g, 98%).

¹H-NMR (300 MHz, DMSO-d₆) δ 8.78 (s, 1H), 7.41 (s, 1H), 7.37 (s, 1H), 4.00 (s, 3H).

### (1-6) N-(3, 4-dichloro-2-fluorophenyl)-7-methoxyquinazolin-6-yloxy)piperidin-1-yl)pro-2-pen-1-one

1.43 g of the compound obtained in (1-5), 1.91 g of (R)-(−)-N-Boc-4-hydroxypiperidine and 1.96 g of triphenylphosphine were added to 20 ml of methylene chloride, and 2.01 ml of diisopropylazodicarboxylate was added thereto dropwise. The resulting mixture was stirred at room temperature for 1 hour and distilled under a reduced pressure, and the residue was briefly purified by column chromatography (ethylacetate:methylenechloride:methanol=20:20:1). The partially purified residue was then dissolved in 60 ml of 2-propanol, 1.17 g of 3,4-dichloro-4-fluoroaniline was thereto, and the mixture was stirred at 100° C. for 3 hours. The resulting mixture was distilled under a reduced pressure to remove the solvent, and the residue was dissolved in 60 ml of methylenechloride. 60 ml of trifluoroacetic acid was added thereto and the mixture was stirred at room temperature for 1 hour. The resulting mixture was distilled under a reduced pressure to remove the solvent. Saturated sodium bicarbonate solution was added to the resulting residue to make it basic, followed by extraction with chloroform. The organic layer was dried over anhydrous sodium sulfate, filtered, and distilled under a reduced pressure. The resulting residue was subjected to column chromatography (chloroform:methanol=1:2) to obtain the title compound.

### Example 2: Preparation of 1-(4-((4-((3,4-dichloro-2-fluorophenyl)amino)-7-methoxyquinazolin-6-yl)oxy)piperidin-1-yl)-2,2-difluorobutene-1,3-dione

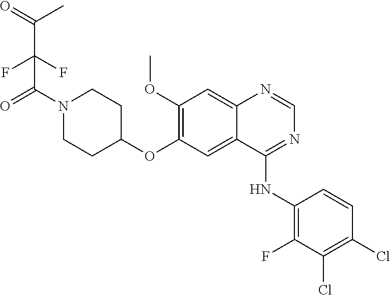 [SMILES: COc1cc2ncnc(Nc3ccc(Cl)c(Cl)c3F)c2cc1OC1CCN(C(=O)C(F)(F)C(C)=O)CC1]

A portion of the compound obtained in (1-6) (1 mmol) is mixed with benzotriazol-1-yl-oxytripyrrolidinophosphonium hexafluorophosphate (PyBop) (1.5 eq), triethylamine (3.0 eq), dichloromethane (30 ml), and 2,2-difluoro-3-oxobutanoic acid (1.3 eq) at room temperature with continued stirring for 12 h.

### Example 3: Preparation of 4-(4-((4-((3,4-dichloro-2-fluorophenyl)amino)-7-methoxyquinazolin-6-yl)oxy)piperidin-1-yl)-N,3,3-trimethyl-2,4-dioxobutanamide

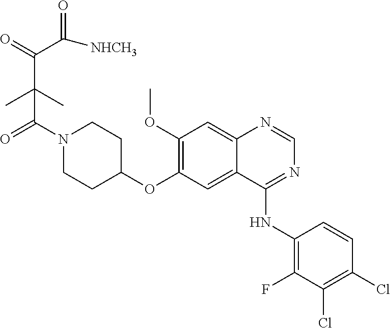 [SMILES: CNC(=O)C(=O)C(C)(C)C(=O)N1CCC(Oc2cc3c(Nc4ccc(Cl)c(Cl)c4F)ncnc3cc2OC)CC1]

A portion of the compound obtained in (1-6) (1 mmol) is mixed with benzotriazol-1-yl-oxytripyrrolidinophosphonium hexafluorophosphate (PyBop) (1.5 eq), triethylamine (3.0 eq), dichloromethane (30 ml), and 2,2-dimethyl-4-(methylamino)-3,4-dioxobutanoic acid (1.3 eq) at room temperature with continued stirring for 12 h.

### Example 4: Preparation of (2-(4-((4-((3,4-dichloro-2-fluorophenyl)amino)-7-methoxyquinazolin-6-yl)oxy)piperidin-1-yl)-2-oxoethyl)boronic acid

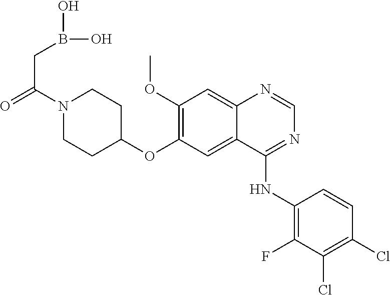 [SMILES: COc1cc2ncnc(Nc3ccc(Cl)c(Cl)c3F)c2cc1OC1CCN(C(=O)CB(O)O)CC1]

A portion of the compound obtained in (1-6) (1 mmol) is mixed with benzotriazol-1-yl-oxytripyrrolidinophosphonium hexafluorophosphate (PyBop) (1.5 eq), triethylamine (3.0 eq), dichloromethane(30 ml), and 2-(di-tert-butoxyboranyl)acetic acid (1.3 eq) at room temperature with continued stirring for 12 h. The mixture is then reacted with concentrated HCl.

### Example 5: Preparation of N1-(2-(dimethylamino)ethyl)-N1-methyl-N4-(4-(1-methyl-1H-indol-3-yl)pyrimidin-2-yl)benzene-1,2,4-triamine

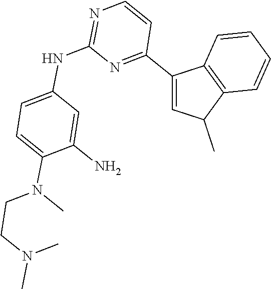 [SMILES: CC1C=C(c2ccnc(Nc3ccc(N(C)CCN(C)C)c(N)c3)n2)c2ccccc21]

2,4-dichloropyrimidine is reacted with MeMgBr (1 eq, 3.2 M in 2-methyl THF) and 1 eq of indole in THF at 0° C. and warmed to 60° C. 1.05 eq sodium hydroxide and 1.05 eq methyl iodide are added at 0° C. to replace the hydrogen on the indole nitrogen with a methyl group. 4-fluoro-5-nitroaniline (1.05 eq), tosic acid (1.1 eq) and 2-pentanol are added to the mixture at 125° C. Subsequently, 2.2 eq of the N,N,N′-trimethylethane-1,2-diamine in DMA are added at 140° C., followed by 3 eq of iron, 0.7 eq of ammonium chloride, ethanol and water at 100° C. to provide the compound of Example 5.

### Example 6: Preparation of (2-((2-((2-(dimethylamino)ethyl)(methyl)amino)-5-((4-(1-methyl-1H-indol-3-yl)pyrimidin-2-yl)amino)phenyl)amino)-2-oxoethyl)boronic acid

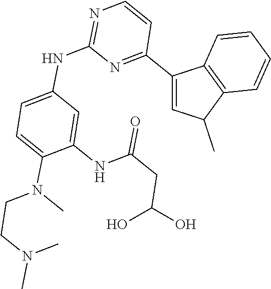 [SMILES: CC1C=C(c2ccnc(Nc3ccc(N(C)CCN(C)C)c(NC(=O)CC(O)O)c3)n2)c2ccccc21]

A portion of the compound obtained in Example 5 (1 mmol) is mixed with benzotriazol-1-yl-oxytripyrrolidinophosphonium hexafluorophosphate (PyBop) (1.5 eq), triethylamine (3.0 eq), and 2-(di-tert-butoxyboranyl)acetic acid (1.3 eq). The mixture is then reacted with concentrated HCl with stirring for 12 hours.

### Example 7: Preparation of N-(2-((2-(dimethylamino)ethyl)(methyl)amino)-5-((4-(1-methyl-1H-indol-3-yl)pyrimidin-2-yl)amino)phenyl)-2,2-difluoro-3-oxobutanamide

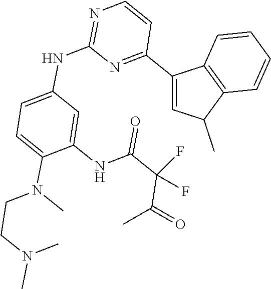 [SMILES: CC(=O)C(F)(F)C(=O)Nc1cc(Nc2nccc(C3=CC(C)c4ccccc43)n2)ccc1N(C)CCN(C)C]

A portion of the compound obtained in Example 5 (1 mmol) is mixed with benzotriazol-1-yl-oxytripyrrolidinophosphonium hexafluorophosphate (PyBop) (1.5 eq), triethylamine (3.0 eq), and 2,2-difluoro-3-oxobutanoic acid (1.3 eq) at room temperature with stirring for 12 h.

### Test Example 1: Inhibition of EGFR Enzyme

10 μl of an EGFR (EGFR type 1 kinase, UPSTATE, 10 ng/μl) is added to each well of a 96-well plate. As an EGFR inhibitor, 10 μl of a serially diluted solution of each of the compounds obtained in Examples 2 to 7, Iressa (Astrazeneca) and Lapatinib (GlaxoSmithKline) is added to each well, and the plate is incubated at room temperature for 10 mins. 10 μl of Poly (Glu, Tyr) 4:1 (Sigma, 10 ng/ml) and 10 μl of ATP (50 μM) are added thereto to initiate a kinase reaction, and the resulting mixture is incubated at room temperature for 1 hour. 10 μl of 100 mM EDTA is added to each well and stirred for 5 mins to terminate the kinase reaction. 10 μl of 10× anti-phosphotyrosine antibody (Pan Vera), 10 μl of 10×PTK (protein tyrosine kinase) green tracer (Pan Vera) and 30 μl of FP (fluorescence polarization) diluted buffer are added to the reacted mixture, followed by incubating in dark at room temperature for 30 mins. The FP value of each well is determined with VICTORIII fluorescence meter (Perkin Elmer) at 488 nm, (excitation filter) and 535 nm (emission filter), and IC50, the concentration at which 50% inhibition is observed, is determined, wherein the maximum (0% inhibition) value is set at the polarized light value measured for the well untreated with an EGFR inhibitor and the minimum value corresponded to 100% inhibition. The calculation and analysis of 1050 are carried out by using Microsoft Excel.

### Test Example 2: Inhibition of EGFR Mutant Enzyme (C797S)

The procedure of Test Example 1 is repeated except that 10 μl of C797S enzyme (EGFR C797S kinase, UPSTATE) is employed instead of 10 μl of the EGFR.

### Test Example 3: Test of Cancer Cell Growth Inhibition

A lung cancer cell line and a breast cancer cell line having an EGFR C797S mutation or HER2 C805S mutation, are used to test the potency of the compounds of the invention in inhibiting the cancer cell growth using a culture medium, DMEM (Dulbecco's Modified Eagle's Medium) having 4.5 g/1 of glucose and 1.5 g/1 of sodium bicarbonate added and supplemented with 10% FBS (fetal bovine serum).

The cancer cell lines stored in a liquid nitrogen tank are each quickly thawed at 37° C., and centrifuged to remove the medium. The resulting cell pellet is mixed with a culture medium, incubated in a culture flask at 37° C. under 5% CO2 for 2 to 3 days, and the medium is removed. The remaining cells are washed with DPBS (Dulbecco's Phosphate Buffered Saline) and separated from the flask by using Tripsin-EDTA. The separated cells are diluted with a culture medium to a concentration of 100,000 cells/ml. 100 μl of the diluted cell suspension is added to each well of a 96-well plate, and incubated at 37° C. under 5% CO2 for 1 day.

The compounds obtained in Examples 2 to 7 as well as the conventional EGFR inhibitors, Iressa and lapatinib, as positive controls, and afatinib, poziotinib, and osimertinib, as negative controls, are each dissolved in 99.5% DMSO to a concentration of 25 mM. In case that the test compound is not soluble in DMSO, a small amount of 1% HCl is added thereto and treated in a 40° C. water bath for 30 mins until a complete dissolution is attained. The test compound solution is diluted with a culture medium to a final concentration of 100 μM, and then diluted 10 times serially to 10-6 μM (a final concentration of DMSO is less than 1%). The medium is removed from each well of the 96-well plate.

100 μl of a test compound solution is added to each well holding the cultured cells, and the plate is incubated at 37° C. under 5% CO₂ for 72 hours. After removing the medium from the plate, 50 μl of 10% trichloroacetic acid is added to each well, and the plate is kept at 4° C. for 1 hour to fix the cells to the bottom of the plate. The added trichloroacetic acid is removed from each well, the plate is dried, 100 μl of an SRB (Sulforhodamine-B) dye solution is added thereto, and the resulting mixture is reacted for 10 mins. The SRB dye solution is prepared by dissolving SRB in 1% acetic acid to a concentration of 0.4%. After removing the dye solution, the plate is washed with water, and dried. When the dye solution is not effectively removed by water, 1% acetic acid is used. 150 μl of 10 mM trisma base is added to each well, and the absorbance at 540 nm is determined with a microplate reader.

IC50, the concentration at which 50% inhibition occurs, is evaluated based on the difference between the final concentration of the test cells and the initial concentration of the cells incubated in a well not-treated with the test compound which is regarded as 100%. The calculation of IC50 is carried out by using Microsoft Excel.

### Test Example 4: Prolongation Study

A lung cancer cell line having the EGFR C797S mutation is used to test the potency of the compounds of the disclosure in inhibiting EGFR's phosphorylation and the prolongation of ability to inhibit it thereof.

The cell line is incubated in a culture flask at 37° C. under 95% air and 5% CO2 using a culture medium containing DMEM, 10% FBS and 1% PS. When more than 90% of the total volume of the culture flask becomes filled with cells, the cultured cell suspension is subject to secondary incubation and is poured to each well of a 6-well plate to the extent of 500,000 cells/well. After 24 hrs, the cells are separated from the solution, washed with PBS, and incubated in a culture medium containing DMEM, 0.1% FBS and 1% PS for 16 hrs. The compounds obtained in Examples 2 to 7, and Tarceva as EGFR phosphorylation inhibitors are each added to the cell-containing well to a concentration of 1 μM. After 4 hrs, the cells are separated from the solution, washed 4 times with PBS after every 0, 2, 4 and 8 hrs, and incubated in a culture medium containing DMEM, 0.1% FBS and 1% PS. When each of 0, 8, 24 and 48 hrs passes after the washing, the medium is removed therefrom to terminate the reaction. Just before the completion of the reaction, the cultured cell solution is treated with a 100 ng/ml concentration of EGF (Sigma, Cat No. E99644) for 5 mins to induce the activation of EGFR. After the completion of the reaction, the well plate holding the cultured cells is stored at −70° C. In control groups, the replacement of the medium was performed instead of the addition of the EGFR phosphorylation inhibitor, wherein the induction of EGFR activation using EGF is made only in a positive control group and not made in a negative control group.

For Western blot and enzyme immune measuring (ELISA) methods, the well plate stored at −70° C. is allowed to melt to room temperature, and then protein is extracted from the cells in the well plate using a protein extract buffer. The extraction of the protein is performed as follows: 250 μl of the protein extract buffer (Phosphosafe extraction reagent, Calbiochem, Cat No. 71296-3) comprising protease inhibitor cocktail is added to each cell-containing well, which is stirred at room temperature for 5 mins. The cells are collected using a cell scraper and put in an 1.5 ml tube, which is centrifuged at a speed of 16,000×g for 5 mins. The upper layer thus obtained is separated, of which the protein content was determined by a protein assay kit (Bio-rad, Cat No. 500-0116). The protein extracted is diluted with PBS to a concentration of 0.8 mg/ml.

A human EGFR (py1173) immunoassay kit (Biosource, Cat No. KHR9071) is used in the enzyme immune measuring method. 100 μl of the sample which is diluted by 4 folds with a standard dilution buffer in a kit is added to a strip well, which is incubated at a 4° C. refrigerator overnight. The cultured cells are separated therefrom and washed 4 times with 200 μl of a washing buffer. 100 μl of the primary antibody (rabbit anti-human EGFR [pY1173]) is put to each strip well, incubated at 37° C. for 1 hr, and washed 4 times with 200 μl of a washing buffer. The secondary antibody (anti-rabbit IgG-HRP) is diluted by 100 fold with an HRP dilution buffer in a kit. 100 μl of the dilute is put to each strip well, incubated at 37° C. for 30 mins, and washed 4 times with 200 μl of the washing buffer. 100 μl of an HRP substrate in a kit is put to each strip well and incubated in a darkroom for 10 to 30 mins. 100 μl of a reaction stop solution is added thereto to terminate the reaction, and then, the absorbance at 450 nm was observed.

Electrophoresis and Western blot methods are conducted based on the conventional methods in the following: An LDS buffer is added to each sample, which is allowed to boil at 70° C. for 10 mins. 10 μl of the resulting solution was loaded to a 12-well gel (Nupage 4-12% Bis-tris gel, Invitrogen), followed by 120 volt-electrophoresis in a buffer (MOPS electrophoresis buffer, Invitrogen, Cat No. NP0006-1) for 2 hrs. After the electrophoresis, the resulting protein bands are transferred to a nitrocellulose membrane (Bio-rad, Cat No. 162-0251) in a transfer buffer (Invitrogen, Cat No. NP0001) with 30 volt for 2 hrs. The nitrocellulose membrane transferred is allowed to react with a 3% BSA blocking solution at room temperature for 1-2 hrs to inhibit a non-specific antigen-antibody reaction. The primary antibody diluted with the blocking solution (anti-EGFR (Stressgen, Cat No. CSA330, 1:100 dilution)), anti-pEGFR (Santacruz, Cat No. SC 12351-R, 1: 500 dilution) and anti-β actin (Sigma, Cat No. A1978, 4 μg/ml dilution) are allowed to react with each other at 4° C. overnight, which is washed 4 times with a washing buffer (TBS-T) for each 10 mins. The secondary antibody diluted with the blocking solution (anti-mouse IgG (Chemicon, Cat No. AP124P, 1:5000 dilution)) and anti-rabbit IgG (Chemicon, Cat No. AP132P, 1:5000 dilution) are allowed to react with each other at room temperature for 1 hr, which is washed 5 times with the washing buffer for each 10 mins, followed by coloring using an ECL western blot detection reagent (Amersham, Cat No. RPN2209) and disclosure to Hyperfilm (Amersham, Cat No. RPN2103K) in a darkroom. Protein bands are observed by development of the film.

The compounds of the disclosure show an excellent anticancer activity by effectively inhibiting the activity of the EGFR C797S mutant kinase and the growth of the cell lines with the mutations, as compared with the conventional irreversible EGFR inhibitors, i.e., poziotinib, osimertinib, and afatinib. The compounds of the disclosure show a highly improved inhibition activity against cell lines having the EGFR C797S mutation or HER2 C805S mutation, whereas none of the compounds of the disclosure inhibit the growth of the enzyme of the cell lines without C797S or C805S mutation. Such results are the effect of the reaction with the serine residue of the compounds of the disclosure, relative to conventional irreversible EGFR inhibitors which do not react with serine. Conventional reversible inhibitors inhibit the enzymes and the cell lines with the mutation but are much less effective compared to the compounds of the disclosure.

### Test Example 5: Inhibition of HER2 Enzyme

10 μl of a HER2 (HER2 kinase, ACRO Biosystems, 10 ng/μl) is added to each well of a 96-well plate. As an HER2 inhibitor, 10 μl of a serially diluted solution of each of the compounds obtained in Examples 2 to 7, Iressa (Astrazeneca) and Lapatinib (GlaxoSmithKline) is added to each well, and the plate is incubated at room temperature for 10 mins. 10 μl of Poly (Glu, Tyr) 4:1 (Sigma, 10 ng/ml) and 10 μl of ATP (50 μM) are added thereto to initiate a kinase reaction, and the resulting mixture is incubated at room temperature for 1 hour. 10 μl of 100 mM EDTA is added to each well and stirred for 5 mins to terminate the kinase reaction. 10 μl of 10× anti-phosphotyrosine antibody (Pan Vera), 10 μl of 10×PTK (protein tyrosine kinase) green tracer (Pan Vera) and 30 μl of FP (fluorescence polarization) diluted buffer are added to the reacted mixture, followed by incubating in dark at room temperature for 30 mins. The FP value of each well is determined with VICTORIII fluorescence meter (Perkin Elmer) at 488 nm, (excitation filter) and 535 nm (emission filter), and 1050, the concentration at which 50% inhibition is observed, is determined, wherein the maximum (0% inhibition) value is set at the polarized light value measured for the well untreated with a HER2 inhibitor and the minimum value corresponded to 100% inhibition. The calculation and analysis of 1050 are carried out by using Microsoft Excel.

### Test Example 6: Inhibition of HER2 Mutant Enzyme (C805S)

The procedure of Test Example 5 is repeated except that 10 μl of C805S enzyme (HER2 C805S kinase) is employed instead of 10 μl of the HER2.

The foregoing description is given for clearness of understanding only, and no unnecessary limitations should be understood therefrom, as modifications within the scope of the invention may be apparent to those having ordinary skill in the art.

All patents, publications and references cited herein are hereby fully incorporated by reference. In case of conflict between the present disclosure and incorporated patents, publications, and references, the present disclosure should control.

What is claimed:

1. An EGFR family tyrosine kinase inhibitor, which is a compound represented by Formula (II) or a pharmaceutically acceptable salt or solvate thereof: 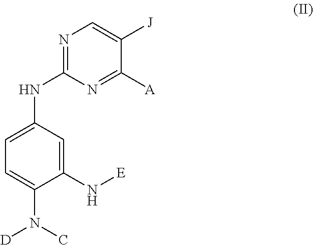
wherein,
A is: 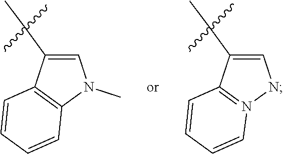
E is: 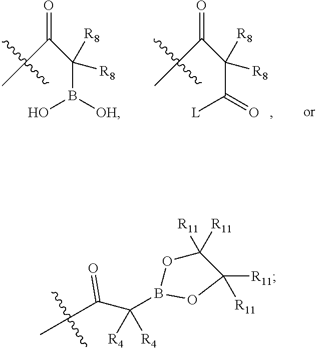
J comprises —CO₂R₁₀, halo, NHC(O)R₁₀;
R₈ are each independently selected from hydrogen, halogen, alkyl, cycloalkyl, perfluoroalkyl, aryl, heteroaryl, or together form cycloalkyl;
R₁₀ comprises hydrogen, halogen, alkyl, cycloalkyl, perfluoroalkyl, aryl, or heteroaryl;
R₁₁ are each independently selected from hydrogen, alkyl, alkyl-CO₂R₁₂, or can together form (═O), and Rig is selected from hydrogen or C₁₋₆ alkyl;
C and D are each independently selected from alkyl, —N(R₈)₂, —OR₈, alkyl-W, or together can comprise a cycloalkyl;
W is selected from —N(R₈)₂ or —OR₈; and
L is selected from —CO₂NH₂, —CO₂NHR₁₀, alkyl, perfluoroalkyl, or cycloalkyl.

2. The EGFR family tyrosine kinase inhibitor of claim 1, wherein one or both of C and D are C₁₋₆ alkyl or together comprise a C₃₋₇ cycloalkyl.

3. The EGFR family tyrosine kinase inhibitor of claim 1, wherein one or both of C and D are substituted with C₁₋₃ alkyl on one or more carbon atoms.

4. The EGFR family tyrosine kinase inhibitor of claim 1, wherein R₈ are each independently selected from C₁₋₆ alkyl, C₃₋₇ cycloalkyl, or together form C₃₋₇ cycloalkyl.

5. The EGFR family tyrosine kinase inhibitor of claim 1, wherein one or both R8 are substituted with C₁₋₃ alkyl on one or more carbon atoms.

6. The EGFR family tyrosine kinase inhibitor of claim 1, wherein L is C₁₋₈ alkyl, C₁₋₈ perfluoroalkyl, or C₃₋₇ cycloalkyl and are unsubstituted or substituted with C₁₋₃ alkyl on one or more carbon atoms.

7. The EGFR family tyrosine kinase inhibitor of claim 1, wherein
E is

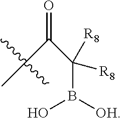

8. The EGFR family tyrosine kinase inhibitor of claim 1, wherein
E is

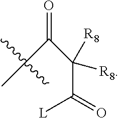

9. The EGFR family tyrosine kinase inhibitor of claim 1, wherein
E is

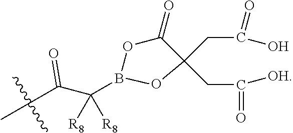

10. The EGFR family tyrosine kinase inhibitor of claim 1, wherein J comprises —CO₂R₁₀.

11. The EGFR family tyrosine kinase inhibitor of claim 1, wherein J comprises —NHC(O)R₁₀.

12. The EGFR family tyrosine kinase inhibitor of claim 1, wherein R₁₀ comprises C₁₋₆ alkyl or C₃₋₇ cycloalkyl.

13. The EGFR family tyrosine kinase inhibitor of claim 1, wherein J is chloro.

14. The EGFR family tyrosine kinase inhibitor of claim 1, selected from the group consisting of:
1) (2-((2-((2-(dimethylamino)ethyl)(methyl)amino)-5-((4-(1-methyl-1H-indol-3-yl)pyrimidin-2-yl)amino)phenyl)amino)-2-oxoethyl)boronic acid; and
2) N-(2-((2-(dimethylamino)ethyl)(methyl)amino)-5-((4-1-methyl-1H-indol-3-yl)pyrimidin-2-yl)amino)phenyl)-2,2-difluoro-3-oxobutanamide.

15. A pharmaceutical composition comprising the EGFR family tyrosine kinase inhibitor compound or its pharmaceutically acceptable salt or solvate of claim 1 as an active ingredient and a pharmaceutically acceptable carrier.

16. A method of treating a cancer in a subject having an EGFR C797S mutation or an HER2 C805S mutation comprising administering to the subject a pharmaceutically effective amount of an EGFR family tyrosine kinase inhibitor compound or its pharmaceutically acceptable salt or solvate according to claim 1.

17. The method of claim 16, wherein the cancer is lung cancer or breast cancer.

18. The method of claim 16, wherein the cancer is breast cancer.

19. The method of claim 16, wherein the subject has the EGFR C797S mutation.

20. The method of claim 16, wherein the subject has the HER2 C805S mutation.
